(19) United States
(12) Patent Application Publication
(10) Pub. No.: US 2023/0355220 A1
(43) Pub. Date: Nov. 9, 2023

(54) CUTTING INSTRUMENT WITH SAFETY SLEEVE

(71) Applicant: Mark Jessup, Northumberland (GB)

(72) Inventor: Mark Jessup, Northumberland (GB)

(21) Appl. No.: 18/246,466

(22) PCT Filed: Sep. 7, 2021
(86) PCT No.: PCT/GB2021/052295

§ 371 Date: Mar. 23, 2023

(30) Foreign Application Priority Data
Sep. 24, 2020 (GB) 2015085.0

(51) Int. Cl. A61B 10/02

(52) U.S. Cl. CPC A61B 10/0266

(57) ABSTRACT

A surgical cutting instrument comprises a handle portion and an elongate shaft. The shaft has a distal end and a proximal end and a longitudinal axis extending therebetween, and a cutting element is mounted at the distal end. The instrument further comprises a safety sleeve which includes a substantially cylindrical channel through which the elongate shaft extends. The safety sleeve is moveable axially along the shaft between the proximal end and the distal end of the shaft. The instrument further comprises locking means for preventing further axial movement of the safety sleeve with respect to the elongate shaft when the safety sleeve is in the extended position. The locking means comprises first and second cooperating elements each located on a respective one of the distal end of the shaft and the safety sleeve. A method for using the cutting instrument is also provided.

### CROSS-REFERENCE TO RELATED APPLICATION(S)

This application is a Section 371 National Stage Application of International Application No. PCT/GB2021/052295, filed Sep. 7, 2021, entitled “CUTTING INSTRUMENT WITH SAFETY SLEEVE”, which claims priority to GB Application No. 2015085.0, filed Sep. 24, 2020, which are incorporated herein by reference in their entirety.

### TECHNICAL FIELD

The present disclosure relates to surgical instruments including a sharp cutting tip which include a safety sleeve for protecting the cutting tip of the instrument after use.

### BACKGROUND

Cutting instruments used in certain surgical procedures, such as biopsy punches or laparascopic scissors, have a sharp cutting tip to allow for the cutting of tissues and/or removal of tissue samples from patients. Needlestick and other sharps injuries are a serious hazard to health professionals, particularly once the equipment has been contaminated with potentially hazardous biological material.

It would be desirable to provide a cutting instrument which includes an integrated safety sleeve to shield the cutting tip after use.

### SUMMARY

According to a first aspect of the disclosure there is provided a surgical cutting instrument comprising a handle portion and an elongate shaft, the shaft having a distal end and a proximal end and a longitudinal axis extending therebetween, the shaft mounting a cutting element at the distal end, wherein the shaft is rotatably mounted onto the handle portion at the proximal end, allowing for rotation of the shaft about the longitudinal axis, the instrument further comprising a safety sleeve including a substantially cylindrical channel through which the elongate shaft extends, wherein the safety sleeve is moveable axially along the shaft between the proximal end and the distal end of the shaft, and wherein the instrument further comprises locking means for preventing further axial movement of the safety sleeve with respect to the elongate shaft when the safety sleeve is in the extended position; The locking means comprises first and second cooperating elements each located on a respective one of the distal end of the shaft and the safety sleeve.

The surgical cutting instrument may be selected from the group comprising: biopsy apparatus and laparoscopic scissors.

The locking means automatically engages when the safety sleeve is moved over the cutting tip. Once the locking means has engaged the safety sleeve cannot then be retracted to expose the cutting tip. The locking means is engaged when the first and second cooperating elements interact.

Preferably, when the safety sleeve is in the retracted position, rotation of the safety sleeve is configured to rotate the elongate shaft.

Preferably the instrument further comprises means for maintaining a static connection between the safety sleeve and the elongate shaft when the safety sleeve is in the retracted position, and preferably the means for maintaining a static connection comprises third and fourth cooperating elements each located on a respective one of the proximal end of the shaft and the safety sleeve.

Preferably, the third and fourth cooperating elements comprise male/female cooperating elements. More preferably, one of the third and fourth cooperating elements is a protrusion and the other of the third and fourth cooperating elements is a slot shaped to accommodate the protrusion.

The first cooperating element may comprise a collar located at the distal end of the elongate shaft, adjacent the cutting tip and the second cooperating element may comprise at least one resilient tab located on the safety sleeve and an annular channel shaped to receive the collar located within the cylindrical channel of the safety sleeve.

Preferably the safety sleeve includes a plurality of resilient tabs.

Preferably, the at least one resilient tab includes a protrusion and one edge of the annular channel is defined by the protrusion.

Alternatively, the first cooperating element may comprise at least one resilient clip including a protrusion located on the safety sleeve and biased towards the elongate shaft; and the second cooperating element may comprise at least one aperture for receiving the or each protrusion located at the distal end of the elongate shaft, adjacent the cutting tip, such that in the extended position the or each protrusion locates within the or each aperture preventing further axial movement of the safety sleeve with respect to the elongate shaft.

Preferably, the safety sleeve includes a plurality of resilient clips, each including a protrusion.

Preferably, the safety sleeve further comprises a portion of increased diameter located adjacent the proximal end of the elongate shaft. Preferably, the portion of the safety sleeve with increased diameter is configured to be gripped and includes a plurality of ridges.

Preferably, the surgical cutting instrument is for extracting a biopsy sample.

According to a second aspect of the disclosure, there is provided a method of using a surgical cutting instrument as hereinbefore defined, with the safety sleeve located in the retracted position, the method comprising the steps of:
ensuring the safety sleeve of the surgical instrument is located in the retracted position;
operating the cutting tip; and
moving the safety sleeve along the elongate shaft towards the cutting tip until the cutting tip is covered by the safety sleeve and the locking means is engaged.

The elongate shaft may be rotated to orient the cutting tip in the desired direction before operating the cutting tip.

Where the instrument is for extracting a biopsy sample the method includes the step of removing a tissue sample after step b).

The disclosure provides an improved surgical cutting instrument which includes an integrated locking safety sleeve to protect users from accidental sharps injuries. By integrating the safety sleeve into the apparatus, the risk of injury through accidental contact with the used cutting tip is minimized.

### BRIEF DESCRIPTION OF THE DRAWINGS

In the Drawings, which illustrate preferred embodiments of cutting apparatus for according to the disclosure, and are by way of example:

FIG. 1 illustrates a biopsy apparatus according to a first embodiment of the disclosure, with a safety sleeve in a retracted position;

FIG. 2 illustrates the biopsy apparatus of FIG. 1 with the safety sleeve in an extended position;

FIG. 3 is an exploded view of the proximal end of the apparatus of FIG. 1, illustrating the connection between the safety sleeve and the elongate shaft of the apparatus;

FIG. 4 illustrates a longitudinal cross-sectional view through the safety sleeve of the apparatus of FIG. 1, with the safety sleeve in a retracted position;

FIG. 5 illustrates a longitudinal cross-sectional view through the cutting tip of the apparatus of FIG. 1;

FIG. 6 illustrates a cross-sectional view through the safety sleeve of the apparatus of FIG. 2, with the safety sleeve in the extended position;

FIG. 7 illustrates a cross-sectional view through the safety sleeve of an alternative embodiment of the apparatus of the disclosure, with the safety sleeve in the retracted position;

FIG. 8 is a close up view of the cutting tip at the distal end of the apparatus of FIG. 7; and

FIG. 9 illustrates a cross-sectional view through the safety sleeve of the apparatus of FIG. 7, with the safety sleeve in the extended position.

### DETAILED DESCRIPTION OF THE PREFERRED EMBODIMENTS

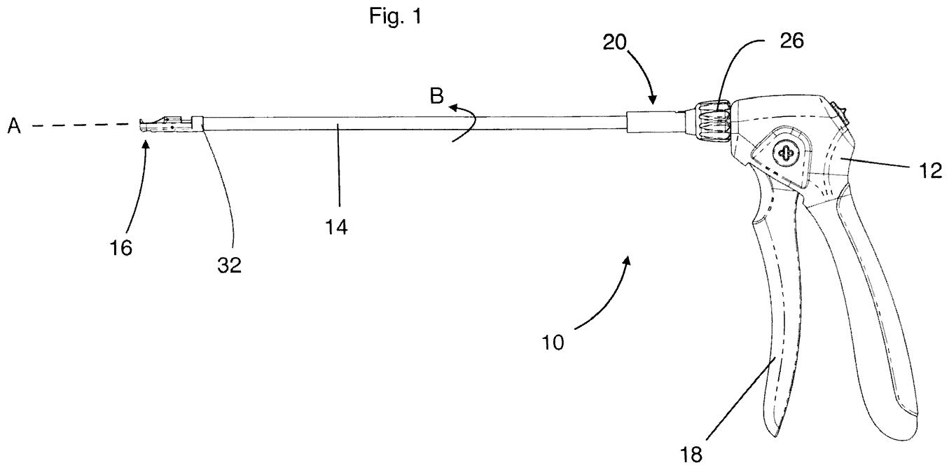

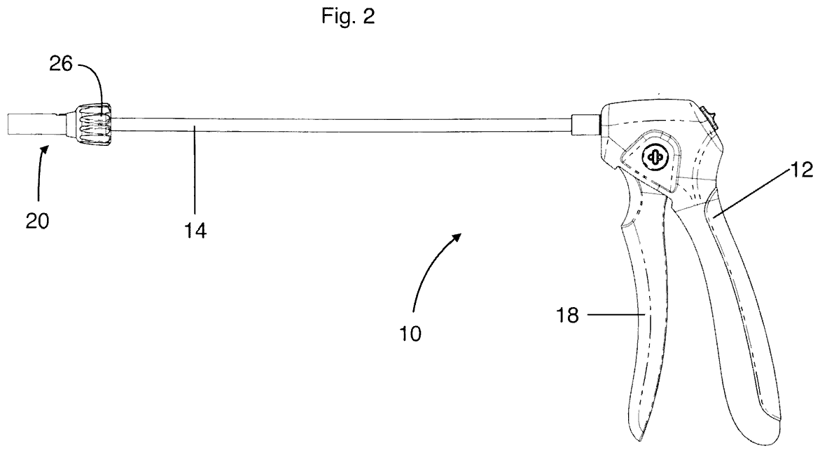

Referring now to FIGS. 1 and 2, a biopsy apparatus is shown generally at 10 comprises a handle portion 12 and an elongate shaft 14 connected to the handle 12 at the proximal end of the shaft 14. A cutting tip 16 is mounted at the distal end of the shaft 14. The distal end of the shaft 14 is the end of the shaft 14 that is furthest away from the handle. The proximal end of the shaft 14 is the end of the shaft 14 that is closest to the handle. The elongate shaft 14 is rotatably mounted on the handle portion 12 so that it may be rotated about its longitudinal axis (labelled ‘A’ in FIG. 1) as shown generally by the arrow marked B in FIG. 1. Rotation of the elongate shaft 14 allows the health professional to correctly orient the cutting tip 16 of the apparatus 10 during the biopsy procedure. The cutting tip 16 is operated using the trigger 18 on the handle portion 12. The mechanism for operation of the cutting tip 16 would be well known to the skilled person.

The apparatus further comprises a safety sleeve 20 which includes a substantially cylindrical channel through which the elongate shaft 14 is mounted. The safety sleeve 20 can be moved axially along the longitudinal axis of the shaft 14 between the proximal and distal ends of the shaft 14. FIG. 1 illustrates the safety sleeve 20 in a retracted position, located at the proximal end of the shaft 14. FIG. 2 illustrates the safety sleeve 20 in an extended position, covering the cutting tip 16 at the distal end of the shaft 14. The safety sleeve 20 is moved between the retracted and extended position manually.

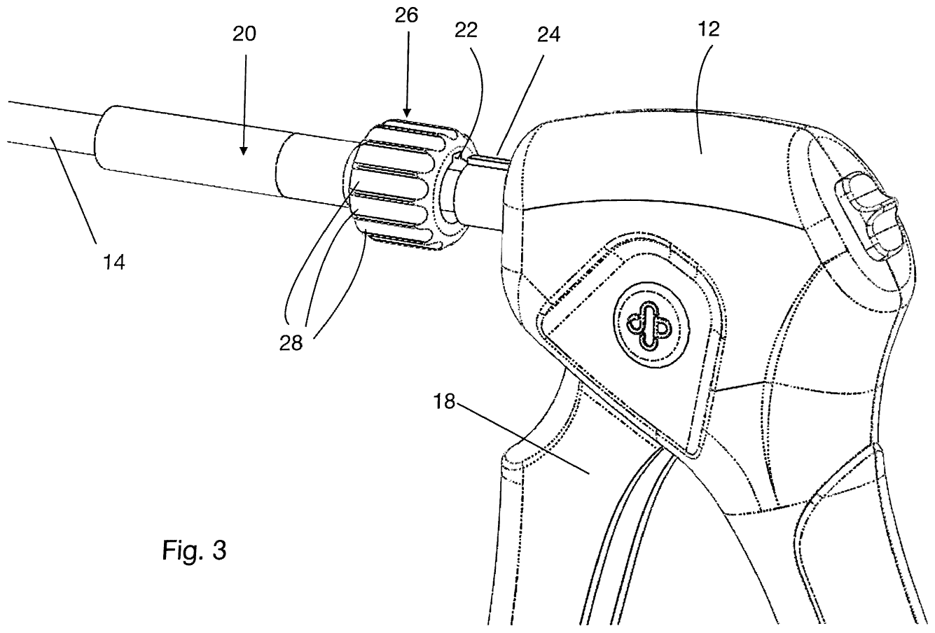

As shown in FIG. 3, the safety sleeve 20 and base of the shaft 14 include cooperating means, in the illustrated example the cooperating means is in the form of a slot 22 on the safety sleeve and a correspondingly shaped protrusion 24 at the proximal end of the shaft 14. The cooperating means ensures a static connection between the shaft 14 and the safety sleeve when the safety sleeve 20 is in the retracted position, and this allows the shaft 14 to be rotated by a user manually gripping and rotating the safety sleeve 20. An alternative combination of male/female connectors could be used to achieve the same result. Preferably, as shown in the figures, the safety sleeve 20 include a base portion 26 of increased diameter, and preferably includes a ribbed surface 28 for enhanced grip. The increased diameter of the base portion 26 provides an easily grippable portion of the sleeve 20 that can be used to rotate the shaft 14, and also serves as a shield to protect the fingers of the user when the safety sleeve is moved along the shaft towards the cutting tip.

The apparatus 10 further includes a locking mechanism to prevent retraction of the safety sleeve 20 once it has been moved to the extended position, covering the cutting tip, as shown in FIG. 2.

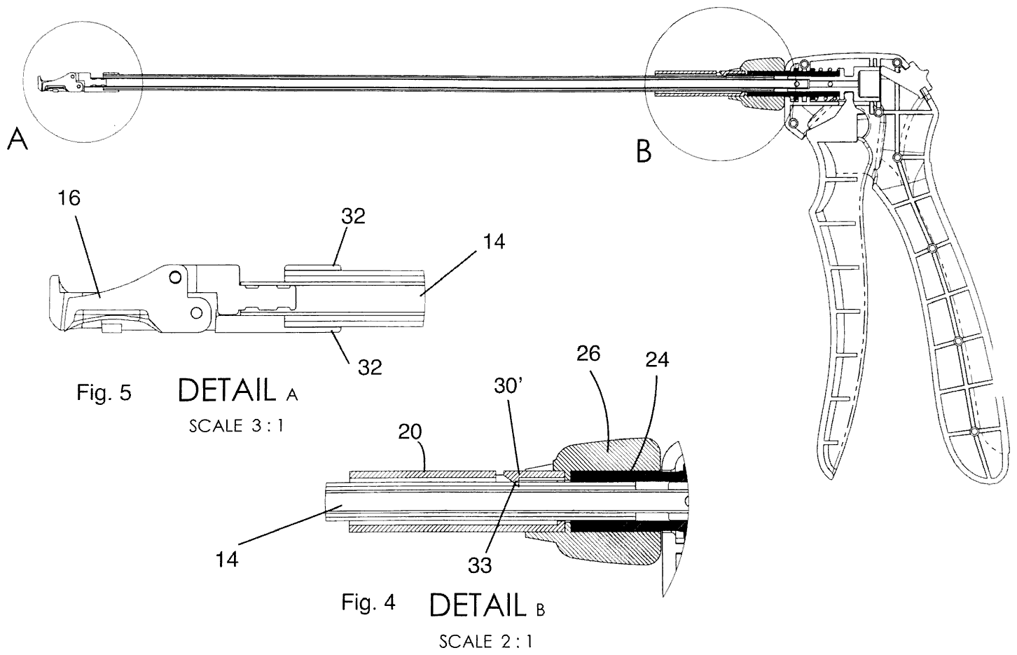

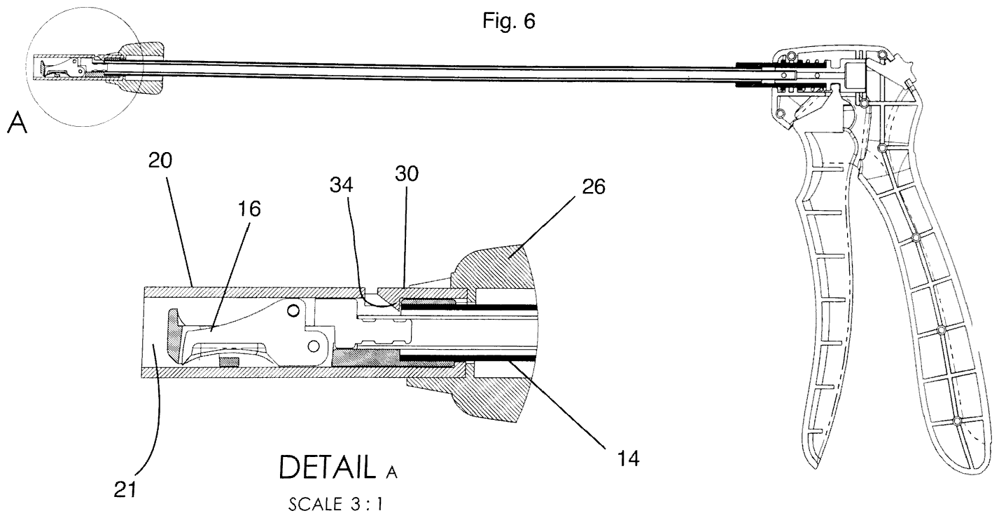

FIG. 4 illustrates a cross-section through the safety sleeve 20 in the retracted position. Internally, the safety sleeve 20 comprises a substantially cylindrical channel 21 shaped to accommodate the elongate shaft 14, along with plurality of resilient clips 30 which are biased towards the shaft 14 when the safety sleeve 20 is in the retracted position. As shown in FIG. 5, the distal end of the elongate shaft 14 includes a collar 32 which has increased diameter compared to the remainder of the shaft 14. The collar 32 is located adjacent the cutting tip. The clip or clips 30 have an enlarged end in the form of a lip 33. After the apparatus 10 has been used to remove a biopsy sample from a patient, the health professional slides the safety sleeve 20 along the elongate shaft 14 towards the cutting tip 16. The resilient clips 30 ride over the collar 32, coming to rest on the distal side of the collar 32. As shown in FIG. 6, the lip 33 of the clip then rests on the distal side of the collar 32, preventing retraction of the safety sleeve back along the elongate shaft. The collar 32 also prevents complete removal of the safety sleeve 20 from the elongate shaft, since the base of the sleeve butts up against the other side of the ridge. By locking the safety sleeve 20 in position over the cutting tip, the health professional is protected from any accidental contact with the cutting tip.

Any means allowing cooperation between the distal end of the elongate shaft and the safety sleeve which enables the sleeve to be locked into position over the cutting tip fulfills the object of the disclosure. This could include a combination of resilient clips on the sleeve and an aperture on the shaft into which the clips, or protrusions on the clips locate.

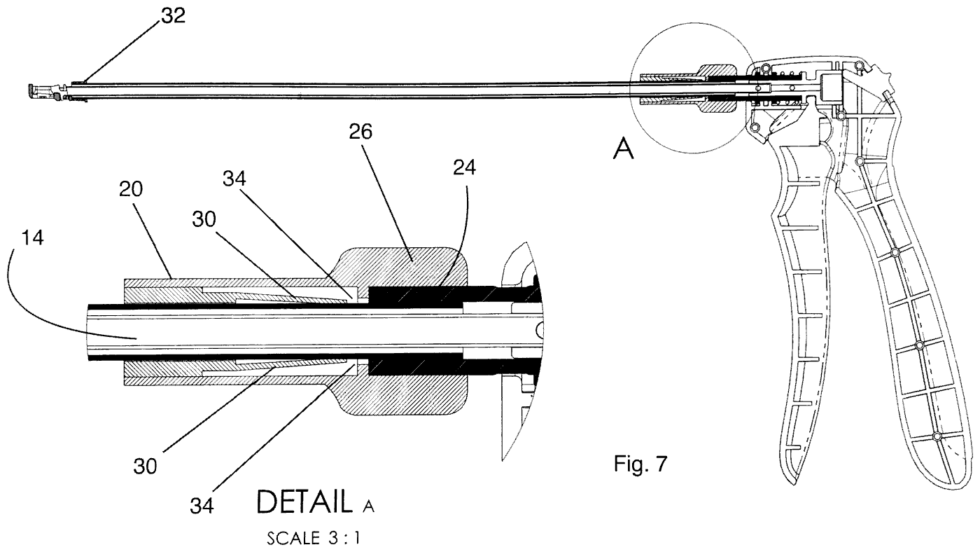

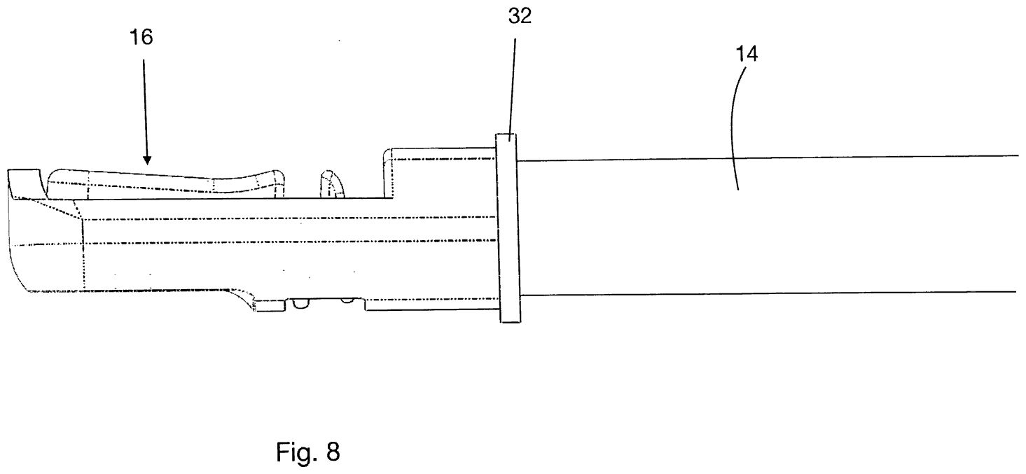

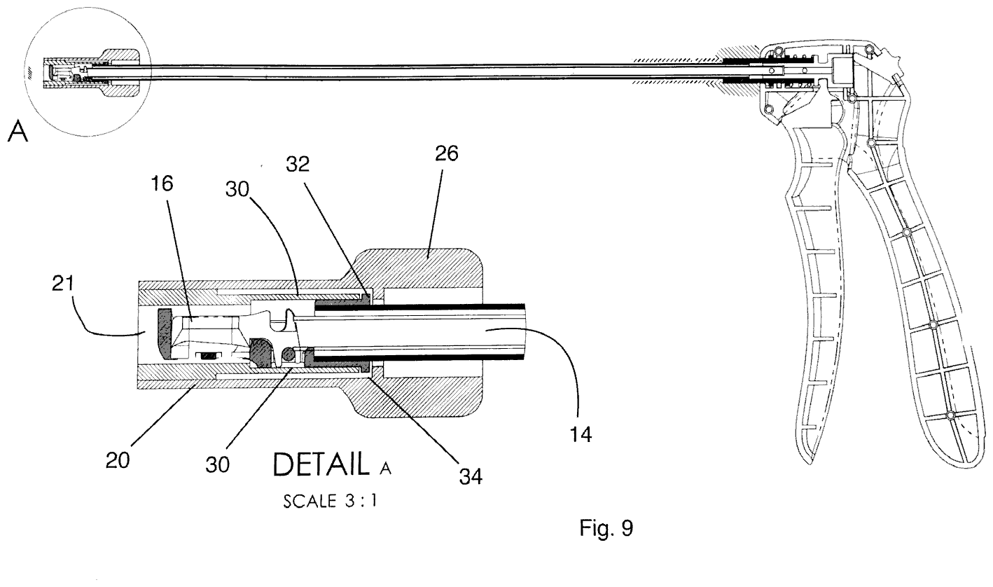

FIG. 7 illustrates a cross-section through an alternative embodiment of the disclosure with the safety sleeve 20 in the retracted position. As with the previous example, internally, the safety sleeve 20 comprises a substantially cylindrical channel 21 shaped to accommodate the elongate shaft 14, however in this example the locking mechanism comprises a plurality of resilient tabs 31 which are biased towards the shaft 14 when the safety sleeve 20 is in the retracted position. As shown in FIG. 8, the distal end of the elongate shaft 14 includes a narrow collar 32′ which has increased diameter compared to the remainder of the shaft 14. The collar 32′ is located adjacent the cutting tip. Internally, as shown in FIG. 7, the safety sleeve 20 also comprises an annular groove or channel 34 shaped to receive the collar 32′ of the shaft 14. After the apparatus 10 has been used to remove a biopsy sample from a patient, the health professional slides the safety sleeve 20 along the elongate shaft 14 towards the cutting tip 16. The resilient tabs 31 ride over the collar 32′, coming to rest on the distal side of the ridge 32′. The collar 32′ then comes to rest in the channel 34, as shown in FIG. 9 and the safety sleeve 20 is now prevented from axial movement along the shaft 14 in either direction. By locking the safety sleeve 20 in position over the cutting tip, the user is protected from any accidental contact with the cutting tip and subsequent injury.

The lockable safety sleeve arrangement could be used in conjunction with any surgical cutting instrument which includes a cutting element at the end of an elongate shaft.

1. A surgical cutting instrument comprising a handle portion and an elongate shaft, the shaft having a distal end and a proximal end and a longitudinal axis extending therebetween, the shaft mounting a cutting element at the distal end, the instrument further comprising a safety sleeve including a substantially cylindrical channel through which the elongate shaft extends, wherein the safety sleeve is moveable axially along the shaft between a retracted position in which a cutting tip is exposed, and an extended position in which the cutting tip is located within the cylindrical channel of the safety sleeve;
wherein the instrument further comprises locking means for preventing further axial movement of the safety sleeve with respect to the elongate shaft when the safety sleeve is in the extended position; and
wherein the locking means comprises first and second cooperating elements each located on a respective one of the distal end of the shaft and the safety sleeve.

2. A surgical cutting instrument according to claim 1, wherein the shaft is rotatably mounted onto the handle portion at the proximal end, allowing for rotation of the shaft about the longitudinal axis.

3. A surgical cutting instrument according to claim 2, wherein, when the safety sleeve is in the retracted position, the safety sleeve is configured to rotate the elongate shaft.

4. A surgical cutting instrument according to claim 2, wherein the instrument further comprises means for maintaining a static connection between the safety sleeve and the elongate shaft, and wherein the means for maintaining a static connection comprises third and fourth cooperating elements each located on a respective one of the proximal end of the shaft and the safety sleeve.

5. A surgical cutting instrument according to claim 4, wherein rotation of the safety sleeve effects rotation of the elongate shaft when the safety sleeve is in the retracted position

6. A surgical cutting instrument according to claim 4, wherein the third and fourth cooperating elements comprise male/female cooperating elements.

7. A surgical cutting instrument according to claim 6, wherein one of the third and fourth cooperating elements is a protrusion and the other of the third and fourth cooperating elements is a slot shaped to accommodate the protrusion.

8. A surgical cutting instrument according to claim 1, wherein the first cooperating means comprises:
a collar located at the distal end of the elongate shaft, adjacent the cutting tip;
at least one resilient tab located on the safety sleeve; and
an annular channel shaped to receive the collar located within the cylindrical channel of the safety sleeve.

9. A surgical cutting instrument according to claim 8, wherein the at least one resilient tab includes a protrusion and one edge of the annular channel is defined by the protrusion.

10. A surgical cutting instrument according to claim 1, wherein the first cooperating element comprises at least one resilient clip including a protrusion located on the safety sleeve and biased towards the elongate shaft; and the second cooperating element comprises at least one aperture for receiving the or each protrusion located at the distal end of the elongate shaft, adjacent the cutting tip, such that in the extended position the or each protrusion locates within the or each aperture preventing further axial movement of the safety sleeve with respect to the elongate shaft.

11. A surgical cutting instrument according to claim 1, wherein the safety sleeve further comprises a portion of increased diameter located adjacent the proximal end of the elongate shaft.

12. A surgical cutting instrument according to claim 11, wherein the portion of the safety sleeve with increased diameter is configured to be gripped and includes a plurality of ridges.

13. A surgical cutting instrument according to claim 1, configured to be used for extracting a biopsy sample.

14. A method of using a surgical cutting instrument according to claim 1, the method comprising the steps of:
a) ensuring the safety sleeve is located in the retracted position;
b) operating the cutting tip; and
c) moving the safety sleeve along the elongate shaft towards the cutting tip until the cutting tip is covered by the safety sleeve and the locking means is engaged.

15. A method of using a surgical cutting instrument according to claim 14, wherein the instrument is configured to be used for extracting a biopsy sample, and the method further includes a step of removing a tissue sample after step b).
